(19) United States
(12) Patent Application Publication
(10) Pub. No.: US 2005/0245910 A1
(43) Pub. Date: Nov. 3, 2005

(54) Tactile feedback finger tip device

(76) Inventors: David Walter Wright, Littleton, CO (US); Lee William Travis, Littleton, CO (US)

Correspondence Address: Bausch & Lomb Incorporated, One Bausch & Lomb Place, Rochester, NY, 14604-2701, US

(21) Appl. No.: 10/835,038

(22) Filed: Apr. 29, 2004

(51) Int. Cl. A61B 17/00

(52) U.S. Cl. 606/1

(57) ABSTRACT

Tactile feedback fingertip device 10 is attached to a surgical handpiece 14 and connected to a control console 24 for providing tactile feedback to a user of a surgical function or parameter.

### BACKGROUND OF THE INVENTION

1. Field of the Invention

The present invention is related to surgical handpieces and more specifically, to surgical handpieces including tactile feedback devices to provide the user with information regarding a particular surgical function.

2. Description of Related Art

In present day surgery, such as ophthalmic surgery, the surgeon is provided with information regarding various surgical functions and parameters during surgery. This feedback enables the surgeon to monitor surgical conditions during surgery and to adjust as necessary to avoid complications. Such warning devices or feedback devices include displays on a surgical console, audio feedback in the form of pre-recorded voice information, or beeps or buzzers. The feedback also includes visual information, such as flashing lights. The surgeon is also provided with tactile feedback through foot pedals, which inform the surgeon as to a foot pedal position, through such mechanisms as detents, springs, or electrical motors to provide resistance of varying levels at different foot pedal positions. However to date, the surgical handpieces themselves have been free from any sort of feedback devices. It would therefore be advantageous to provide tactile feedback to a surgeon in a surgical handpiece to provide feedback to a sense, i.e., touch that is relatively underutilized.

### BRIEF DESCRIPTION OF DRAWINGS

FIG. 1 is a perspective view of a surgical handpiece in accordance with the present invention being held by a user;

FIG. 2 is a partial perspective view of a surgical handpiece in accordance with the present invention shown providing feedback to the user; and

FIG. 3 is a system diagram showing a surgical handpiece in accordance with the present invention in use in surgery.

### DETAILED DESCRIPTION OF THE PREFERRED EMBODIMENT

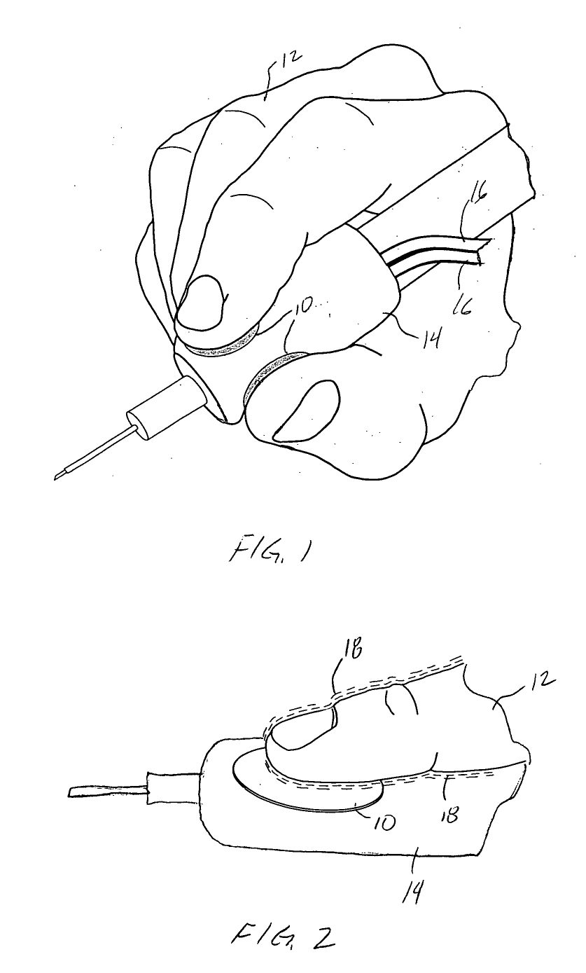

FIG. 1 shows a tactile fingertip device 10, in accordance with the present invention, being held by a user 12. Tactile feedback fingertip device 10 is shown attached to an ophthalmic surgical handpiece 14 for performing a surgical function, such as the phacoemulsification handpiece shown. It is understood that handpiece 14 may be other surgical handpieces, such as vitrectomy cutters, pneumatic scissors, laser probes, or other surgical handpieces commonly known.

Fingertip devices 10 are attached to the handpiece 14 and connected to a control console (not shown) via lines 16. Fingertip devices 10 provide tactile feedback to a user of some surgical function or parameter. Preferably, tactile feedback fingertip devices 10 are well known piezo-resistive elements, which are activated via line 16 by the control console, to warn or inform the user of some surgical function or parameter. Such surgical functions may include at least one or more of the following: loss of irrigation fluid, excessive build-up of vacuum level, surgery time, intraocular pressure level, phaco power, phaco duration, and laser time.

It would also be appreciated that devices 10 may be modulated by the console (not shown) to provide varying frequencies of vibration in order to indicate varying levels of warning to a user. For example, as phaco time increases the vibration of device 10 may also increase.

Another significant advantage of the present invention includes the ability to provide a surgeon, through device 10, with a real-time “feel” for the surgical function being performed by handpiece 14. This concept is similar to fly-by-wire or drive-by-wire systems where the “feel” of the control stick or steering wheel is provided with more movement resistance as the stick or wheel is attempted to be moved more rapidly. This provides a user with a feel for flap resistance or a feel for the road. In the present invention, coordinating the vibration of device 10 with the current surgical conditions would provide the surgeon instantaneous and a more naturally comprehended feedback as to the surgical conditions. For example, in phaco surgery as the tip vibrates and the surgeon increases the phaco power applied to the cataract, device 10 can be made to increase it's frequency of oscillation. Another example is that as the phaco needle transitions from relatively soft tissue to comparatively harder or more dense tissue the handpiece 14 experiences an increased load to maintain the same phaco oscillation. The device 10 can provide the surgeon with instantaneous feedback of this increased load by increasing the amplitude of the vibration of device 10. In this way the surgeon is provided with a “feel” for the current surgical conditions under which the handpiece 14 is operating. Similar “feel” feedback may be applied to other types of handpieces 14 that pertain to the particular surgical function performed by the handpiece 14.

It is noted that line 16, while shown on the outside of device 14 in FIG. 1, may also be incorporated within device 14 in order to be conveniently placed out of the way of user 12.

FIG. 1 shows two (2) devices 10 for providing tactile feedback to the user for two surgical functions; however, it will be appreciated that one or any number of devices 10 may be incorporated into surgical handpiece 14.

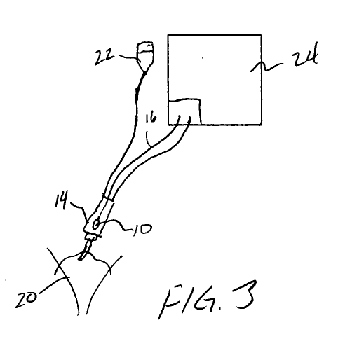

FIG. 2 is a partial perspective view showing a device 10 attached to a handpiece 14 providing tactile feedback to a user's 12 finger tip. The dashed lines 18 indicate vibration of device 10 being transferred to user's 12 finger.

FIG. 3 shows a modified block diagram showing a handpiece 14 with a device 10 attached thereto in use during surgery. Handpiece 14 is performing surgery on an eye 20 and is connected to an irrigation source 22 and a console 24. Device 10 is connected to console via line 16 through which console 24 may activate device 10 to provide the user with a warning or other information concerning surgical functions or parameters. As discussed above, console 24 may also vary the amplitude or frequency of vibration of device 10 to indicate varying levels of warning to the user.

While the present invention has been described in relation to a piezo resistive device, other means of providing tactile feedback may also be used, such as micro-motors, pager vibration motors, a small solenoid, or other devices that can provide tactile feedback to a user.

It is noted that a surgical function or parameter that is indicated by the activation of device 10 may be one of many different parameters and can be preset by console 24 or preferably can be selected by the user amongst a group of parameters. Tactile feedback can also be used to replace or compliment existing audio and visual cues currently employed by present day surgical systems. Preferably, the vibration of device 10 is achieved by a piezo-electric ceramic element coupled to a metal diaphragm (not shown).

1. A tactile feedback fingertip device for a surgical handpiece comprising:
a surgical handpiece for performing a surgical function; and
a fingertip device attached to the handpiece and for connection to a control console for providing tactile feedback to a user of a surgical function or parameter.

2. The fingertip device of claim 1, wherein the device includes one taken from a group consisting of a piezo-electric ceramic element, a micro-motor, a pager motor, or a solenoid for providing a vibrating tactile feedback to the user.

3. The fingertip device of claim 2, wherein a frequency or amplitude of the vibration is varied to indicate varying levels of warning to the user.

4. The fingertip device of claim 2, wherein a frequency or amplitude of the vibration is varied to indicate current surgical conditions.

5. The fingertip device of claim 1, wherein the surgical function is one taken from the group consisting of loss of irrigation fluid, excessive build-up of vacuum level, surgery time, intraocular pressure, phaco power, phaco duration, and laser time.
